(19) United States
(12) Patent Application Publication
(10) Pub. No.: US 2008/0177279 A1
(43) Pub. Date: Jul. 24, 2008

(54) DEPOSITING RADIATION IN HEART MUSCLE UNDER ULTRASOUND GUIDANCE

(75) Inventors: Thilaka Sumanaweera, Los Altos, CA (US); Patrick Maguire, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cyberheart, Inc., Menlo Park, CA (US)

(21) Appl. No.: 11/971,399

(22) Filed: Jan. 9, 2008

Related U.S. Application Data
(60) Provisional application No. 60/879,724, filed on Jan. 9, 2007.
(60) Provisional application No. 60/879,654, filed on Jan. 9, 2007.
(60) Provisional application No. 60/918,540, filed on Mar. 16, 2007.
(60) Provisional application No. 60/975,373, filed on Sep. 26, 2007.

(51) Int. Cl. A61B 19/00

(52) U.S. Cl. 606/130; 901/41

(57) ABSTRACT

A method and system are disclosed for radiosurgical treatment of moving tissues of the heart, including acquiring at least one volume of the tissue and acquiring at least one ultrasound data set, image or volume of the tissue using an ultrasound transducer disposed at a position. A similarity measure is computed between the ultrasound image or volume and the acquired volume or a simulated ultrasound data set, image or volume. A robot is configured in response to the similarity measure and the position of the transducer, and a radiation beam is fired from the configured robot.

### CROSS-REFERENCES TO RELATED APPLICATIONS

This application claims the benefit under 35 USC 119(e) of U.S. Provisional Application No. 60/879,654, filed on Jan. 9, 2007, and entitled “Depositing Radiation In Heart Muscle Under Ultrasound Guidance”, and U.S. Provisional Application No. 60/879,724, filed on Jan. 9, 2007, entitled “Method For Depositing Radiation In Heart Muscle”, the full disclosures of which are incorporated herein by reference.

This application is related to U.S. Provisional Application No. 60/918,540, filed on Mar. 16, 2007, entitled “Radiation Treatment Planning And Delivery For Moving Targets In The Heart”, and U.S. Provisional Application No. 60/975,373, filed on Sep. 26, 2007, entitled “Radiosurgical Ablation of the Myocardium”, the full disclosures of which are incorporated herein by reference.

### BACKGROUND OF THE INVENTION

The present invention is generally related to treatment of the heart, and in particular, non-invasive treatment of the heart using radiosurgical ablation. The present invention generally provides improved methods, devices, and systems for treatment of tissue, in many cases by directing radiation from outside the body toward an internal target tissue. Exemplary embodiments may deposit a specified radiation dose at a target in the heart muscle while minimizing the dose received by adjoining critical structures by using ultrasound images for tracking the target in real time.

In the past, targets such as tumors in the head, spine, abdomen and lungs have been successfully treated by using radiosurgery. During radiosurgery, the target is bombarded with a series of beams of ionizing radiation (for example, a series of MeV X-ray beams) fired from various different positions and orientations by a radiation delivery system. The beams can be directed through intermediate tissue toward the target tissue so as to affect the tumor biology. The beam trajectories help limit the radiation exposure to the intermediate and other collateral tissues, using the cumulative radiation dose at the target to treat the tumor. The CyberKnife™ Radiosurgical System (Accuray Inc.) and the Trilogy™ radiosurgical system (Varian Medical Systems) are two such radiation delivery systems.

Modern robotic radiosurgical systems may incorporate imaging into the treatment system so as to verify the position of the target tissue without having to rely on rigid frameworks affixing the patient to a patient support. Some systems also have an ability to treat tissues that move during respiration, and this has significantly broadened the number of patients that can benefit from radiosurgery. It has also previously been proposed to make use of radiosurgical treatments for treatment of other tissues that undergo physiological movements, including the directing of radiation toward selected areas of the heart for treatment of atrial fibrillation.

During atrial fibrillation, the atria lose their organized pumping action. In normal sinus rhythm, the atria contract, the valves open, and blood fills the ventricles (the lower chambers). The ventricles then contract to complete the organized cycle of each heart beat. Atrial fibrillation has been characterized as a storm of electrical energy that travels across the atria, causing these upper chambers of the heart to quiver or fibrillate. During atrial fibrillation, the blood is not able to empty efficiently from the atria into the ventricles with each heart beat. By directing ionizing radiation toward the heart based on lesion patterns used in open surgical atrial fibrillation therapies (such as the Maze procedure), the resulting scar tissue may prevent recirculating electrical signals and thereby diminish or eliminate the atrial fibrillation.

While the proposed radiosurgical treatments of atrial fibrillation offer benefits by significantly reducing trauma for heart patients, improvements to existing radiosurgical systems may be helpful to expand the use of such therapies. For example, movement of the tissues of the heart during a heartbeat may be significantly more rapid than movements of lung tumors induced by respiration. While well suited for treatment of lung tissues and the like, existing systems used to verify target registration may also limit radiation exposure of collateral tissues and/or avoid delays in the procedure by limiting the rate at which x-ray images are acquired during treatment. As several radiation-sensitive structures are in and/or near the heart, and as the treatment time for a single heart patient may be as long as 30 minutes or more, increasing the imaging rate and/or delaying the radiation beams when the target tissue is not sufficiently aligned may be undesirable in many cases.

In light of the above, it would be desirable to provide improved devices, systems, and methods for treating moving tissues of a patient, particularly by directing radiation from outside the patient and into target tissues of a heart. It would be particularly beneficial if these improvements were compatible with (and could be implemented by modification of) existing radiosurgical systems, ideally without significantly increasing the exposure of patients to incidental imaging radiation, without increasing the costs so much as to make these treatments unavailable to many patients, and/or without unnecessarily degrading the accuracy of the treatments and without causing collateral damage to the healthy tissue despite the movement of the target tissues during beating of the heart.

### BRIEF SUMMARY OF THE INVENTION

The present invention generally provides improved medical devices, systems, and methods, particularly for treatment of moving tissues. The invention allows improved radiosurgical treatment of tissues of the heart, often enhancing the capabilities of existing robotic radiosurgical systems for targeting tissues of the heart to mitigate arrhythmias such as atrial fibrillation or the like.

In a first aspect, the invention provides a method for treating a moving tissue. The method comprises acquiring at least one volume of the tissue and acquiring at least one ultrasound image or volume of the tissue using an ultrasound transducer disposed at a position. A similarity measure is computed between the ultrasound image or volume and the acquired volume or a simulated slice therefrom. A robot is configured in response to the similarity measure and the position of the transducer, and a radiation beam is fired from the configured robot.

In another aspect, the invention provides a method for treating a heart. The method comprises acquiring at least one volume of the heart and acquiring at least one ultrasound data set of the heart using a transducer. A simulation of an ultrasound data set is done from the volume by taking into account a depth-dependent- and steering angle-dependent effects of the ultrasound data set. A similarity measure is computed between the ultrasound image and the simulated ultrasound data set. A robot is configured in response to the similarity measure and a radiation beam is fired from the configured robot.

In another aspect, the invention provides a method for treating a target on a moving heart. The method comprises acquiring at least one volume of the target and acquiring at least one ultrasound volume of the target using a volumetric transducer. A similarity measure is computed between the ultrasound volume and the volume. A 6 DOF robot is configured in response to the similarity measure and the position of the transducer, and a radiation beam is fired from the configured robot.

In another aspect, the invention provides a system for treating a moving tissue. The system comprises a volume acquisition system for acquiring at least one volume of the tissue and a transducer with a position sensor for acquiring at least one ultrasound image or volume of the tissue and associated position information. A processor is coupled to the acquisition system and the transducer. The processor is configured for computing a similarity measure between the ultrasound image or volume and the acquired volume. Alternatively, the depth- and steering angle-dependent effects of the ultrasound image or volume is simulated form the acquired volume prior to computing a similarity measure. A configuration is determined in response to the similarity measure and data and the associated position information, and a robot coupled to the processor implements the configuration. The radiation beam source is supported by the robot.

### BRIEF DESCRIPTION OF THE DRAWINGS

FIG. 1 is an exemplary CyberKnife stereotactic radiosurgery system for use in embodiments of the invention.

FIG. 2 is a graph showing exemplary data from the anterior/posterior motion of a point at the cavotricuspid isthmus inside the right atrium of a pig heart.

FIG. 3 is an illustration of an EKG waveform showing exemplary phases where a time-sequence of CT volumes are acquired.

FIG. 4 is an illustration of a bi-planer transesophageal (TEE) ultrasound transducer acquiring ultrasound images of the heart.

FIG. 5 is an illustration of an ultrasound transducer and a lens focusing the ultrasound energy to a geometric focus.

FIG. 6 schematically illustrates a method for treating a target tissue using a radiosurgical system.

FIG. 6A illustrates a refined method based on that of FIG. 6, in which a moving target tissue of the heart is treated using a radiosurgical system that measures heart cycle signals during imaging and treatment.

FIG. 7 schematically illustrates a more detailed functional block diagram of an exemplary treatment system according to an embodiment of the invention.

FIG. 8 illustrates stereotactic radiosurgery using the system of FIG. 1.

### DETAILED DESCRIPTION OF THE INVENTION

The present invention generally provides improved devices, systems, and methods for treatment of tissue, often using radiosurgical systems. The invention is particularly well suited for tracking of moving tissues such as tissues of the heart and tissue structures adjacent the heart that move with the cardiac or heartbeat cycles. The invention may take advantage of structures and methods which have been developed for treating tumors, particularly those which are associated with treatments of tissue structures that move with the respiration cycle. The cardiac cycle is typically considerably faster than the respiration cycle. The overall treatment times can also be quite lengthy for effective radiosurgical procedures on the heart (typically being greater than 10 minutes, often being greater than ½ hour, and in many cases, being two hours or more). Hence, it will often be advantageous to avoid continuous imaging of the target and adjacent tissues using fluoroscopy or the like. A variety of differing embodiments may be employed, with the following description presenting exemplary embodiments that do not necessarily limit the scope of the invention.

Embodiments of the invention may make use of a motion model of a tissue volume encompassing the target tissue. The motion model may be correlated to a heart signal sensor such as an electrocardiogram (ECG) or (EKG). The motion model may be derived by acquiring 3-D volumes while measuring the heart cycle signals, and the heart cycle signals may also be monitored during treatment so as to predict the position of the target tissue. Multiple models may be employed, including separation of the motion model into a cardiac cycle model and a respiration cycle model. In some embodiments, a pre-treatment model may be used for planning and registration. An intra-operative model may be employed to track motion of the heart during treatment, often in response to external fiducials and/or a heart cycle signal.

Radiosurgery is a known method of treating targets in the body, such as tumors in the head, spine, abdomen and lungs. During radiosurgery, the target is bombarded with a series of MeV X-ray beams fired from various different positions and orientations by using a radiation delivery system, to affect the tumor biology using the cumulative radiation dose at the target. The radiation can be delivered invasively in conjunction with traditional scalpel surgery, or through a percutaneous catheter. Radiation can also be delivered non-invasively from outside the body, through overlying tissue. CyberKnife™ (Accuray Inc.) and Trilogy™ (Varian Medical Systems) are two such radiation delivery systems. Advances in stereotactic surgery have provided increased accuracy in registering the position of tissue targeted for treatment and a radiation source. For example, see U.S. Pat. Nos. 6,351,662 and 6,402,762. Stereotactic radiosurgery systems may be commercially available from ACCURAY, INC. of Sunnyvale, Calif., and BRAINLAB. The Accuray Cyberknife™ stereotactic radiosurgery system has reportedly been used to provide targeted, painless, and fast treatment of tumors.

Improvements in imaging and computer technology have led to advances in radiation treatment, often for targeting tumors of the spine and brain. The introduction of CT scanners enables surgeons and radiation oncologist to better define the location and shape of a tumor. Further improvements in imaging technology include MRI and PET scanners. In addition, radiation therapy has also been aided by enhancements in ancillary technologies such as simulators to help position patients and advanced computers to improve treatment planning to enable the radiation oncologist to deliver radiation from a number of different angles. Computer technology has been introduced that enable radiation oncologists to link CT scanners to radiation therapy, making treatment more precise and treatment planning faster and more accurate, thereby making more complex plans available. Such advancements allow integrated conformal therapy, in which the radiation beam conforms to an actual shape of a tumor to minimize collateral damage to the surrounding healthy tissue. By combining simulators and imaging and treatment planning computers, the irradiation can be precisely administered.

The present invention may take advantage of many components included in or derived from known radiation delivery system components. Suitable system components may comprise:
1. A linear accelerator (Linac) capable of generating the X-ray beam
2. A mechanism to position and orient the X-ray beam.
3. A patient registration system to position and orient the target in the coordinate system of the delivery system.
4. A tracking system for tracking the target during treatment in case the target changes shape or moves between the time of, for example, a CT exam and the time of treatment, and/or during treatment.
5. A couch capable of positioning the target (patient) independent of the mechanism described in #2 above.

In exemplary CyberKnife-based systems, the above 5 items may correspond to:
1. A 6 MeV X-band X-ray Linac
2. A 6 degree-of-freedom (DOF) robotic manipulator.
3. A patient registration system consisting of an ultrasound imaging system.

During treatment, ultrasound data sets are acquired and registered with the CT data by cross-correlating the ultrasound data with simulated ultrasound data generated using the CT data, called digitally reconstructed ultrasound (DRUS). The ultrasound transducer may also fitted with a position sensor to detect its location in the room coordinate system.
4. The tracking system may include several light-emitting diodes (LEDs) mounted on the patent's skin to provide additional information.
5. A couch with 5 DOF.

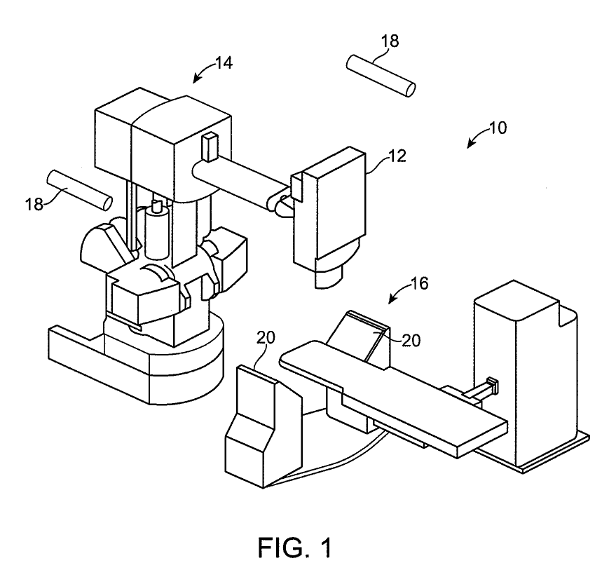

An exemplary Cyberknife stereotactic radiosurgery system 10 is illustrated in FIG. 1. Radiosurgery system 10 has a single source of radiation, which moves about relative to a patient. Radiosurgery system 10 includes a lightweight linear accelerator 12 mounted to a highly maneuverable robotic arm 14. An image guidance system 16 uses image registration techniques to determine the treatment site coordinates with respect to linear accelerator 12, and transmits the target coordinates to robot arm 14 which then directs a radiation beam to the treatment site. When the target moves, system 10 detects the change and corrects the beam pointing in real-time or near real-time. Real-time or near real-time image guidance may avoid any need for skeletal fixation to rigidly immobilize the target.

System 10 makes use of robot arm 14 and linear accelerator 12 under computer control. Image guidance system 16 can monitor patient movement and automatically adjust system 10 to maintain the radiation beam directed at the selected target tissue. Rather than make use of radiosurgery system 10 and related externally applied radiosurgical techniques to tumors of the spine and brain tissues, the invention applies system 10 to numerous cardiac conditions, and in one exemplary method to the treatment of atrial fibrillation (AF).

Tradition radiosurgery instruments without image guidance technology rely on stereotactic metal frames screwed into the patient's skull to accurately target a tumor. Traditional radiosurgery has its drawbacks, the biggest of which relate to the use of the frame, including the pain and difficulty of accurately reattaching the frame in precisely the same location, along with the inability to target tissues other than those in the neck and head. Conventional linear accelerators for these systems can also be the size and weight of an automobile. Frame-based radiosurgery is generally limited to isocentric or spherical target treatments. To allow a device which can precisely pinpoint and treat tissues throughout the body, system 10 makes use of a portable linear accelerator, such as those originally designed for industrial inspections, which can be carried on a person's back. Linear accelerators may be commercially available from SCHONBERG RESEARCH GROUP, SIEMENS, PICKER INTERNATIONAL INC. or VARIAN.

System 10 allows intensity modulated radiation therapy. Using computerized planning and delivery, intensity modulated radiation therapy conforms the radiation to the shape of (for example) a tumor. By using computers to analyze the treatment planning options, multiple beams of radiation match the shape of the tumor. To allow radiosurgery, system 10 can apply intense doses of high-energy radiation to destroy tissue in a single treatment. Radiosurgery with system 10 uses precise spatial localization and large numbers of cross-fired radiation beams. Because of the high dosage of radiation being administered, such radiosurgery is generally more precise than other radiation treatments, with targeting accuracies of 1 to 2 mm.

Linear accelerator 12 is robotically controlled and delivers pin-point radiation to target regions throughout the body of the patient. Radiation may be administered by using a portable linear accelerator such as that illustrated in FIG. 1. Larger linear accelerators may also generate the radiation in some embodiments. Such linear accelerators may be mounted on a large rotating arm that travels around the patient, delivering radiation in constant arcs. This process delivers radiation to the target tissue and also irradiates a certain amount of surrounding tissue. As a result, such radiation therapy may be administered in a series of relatively small doses given daily over a period of several weeks, a process referred to as fractionation. Each radiation dose can create some collateral damage to the healthy surrounding tissue.

In the exemplary embodiment, robot arm 14 of system 10 is part of a pure robotics system, providing six degree of freedom range of motion. In use, the surgeon basically pushes a button and the non-invasive procedure is performed automatically with the image guidance system continuously checking and re-checking the position of the target tissue and the precision with which linear accelerator 12 is firing radiation at the tumor. Image guidance system provides ultrasound guidance that gives the surgeon the position of internal organs. Image guidance system continuously checks, during a procedure, that the radiation beam is directed to the target. Alternatively the image guidance system includes an X-ray imaging system as is the case with the traditional Accuray CyberKnife™ radiosurgery system. The exemplary image guidance system takes the surgeon's hand out of the loop. The surgeon may not even be in the operating room with the patient. Instead, the image guidance system guides the procedure automatically on a real-time basis. By combining advanced image guidance and robotics, system 10 has proven effective in treating head and neck tumors without having to resort to stereotactic metal frame screwed into the skull of a patient.

Image guidance system includes an ultrasound transducer and an ultrasound data acquisition system. The ultrasound transducer is fitted with a position sensor that provides the position and orientation of the transducer with respect to the room coordinate system. The system 10 can determine the location of the target, for example inside the heart, by comparing DRUSs derived from the CT data acquired from the patient with the ultrasound data acquired by the real-time ultrasound imaging system. Image guidance system 16 may also include diagnostic x-ray sources 18 and image detectors 20, this imaging hardware comprising two fixed diagnostics fluoroscopes. These fluoroscopes provide a stationary frame of reference for locating the patient's anatomy, which, in turn, has a known relationship to the reference frame of robot arm 14 and linear accelerator 12. System 10 can determine the location of the skull or spine in the frame of reference of the radiation delivery system by comparing digitally reconstructed radiographs derived from the treatment planning images with radiographs acquired by the real-time imaging systems of the fluoroscopes.

Once the target position is determined, the coordinates are relayed to robot arm 14, which adjusts the pointing of linear accelerator 12 and radiation is delivered. The speed of the imaging process allows the system to detect and adjust to changes in target position in less than one second. The linear accelerator is then moved to a new position and the process is repeated. Alternative systems may make use of laser triangulation, which refers to a method of using so-called laser tattoos to mark external points on the skin's surface so as to target the location of internal organs and critical structures. An alternative system commercialized by BRAINLAB uses a slightly different approach that measures chest wall movements.

System 10 combines robotics and advanced image-guidance to deliver true frameless radiosurgery. Multiple beams of image guided radiation are delivered by robot arm 14 mounted linear accelerator 12. The radiation can converge upon a tumor, destroying it while minimizing exposure to surrounding healthy tissue. Elimination of a stereotactic frame through the use of image guided robotics enables system 10 to treat targets located throughout the body, not just in the head. Radiosurgery is thus possible in areas such as the spine that have traditionally been difficult to treat in the past with radiosurgery, and for pediatric patients such as infants, whose skulls are too thin and fragile to undergo frame-based treatment.

System 10 allows ablation of tissue anywhere in the patient's body. The present invention uses high energy x-ray irradiation from a linear accelerator mounted on a robot arm to produce ablation of target tissue. In one example, system 10 is used to ablate tumors or other defects of the heart treatable with radiation.

Advantages of system 10 include a treatment which can be provided on an outpatient basis, providing a painless option without the risk of complications associated with open surgery. Treatment may be applied in a single-fraction or hypo-fractionated radiosurgery (usually 2 to 5 fractions) for treatment near sensitive structures. System 10 provides flexibility in approach through computer control of flexible robotic arm 14 for access to hard-to-reach locations. System 10 allows isocentric (for spherical) or non-isocentric (for irregularly shaped) target shapes. The creation of the target shapes also takes into account critical surrounding structures, and through the use of robotic arm 14, harm to the critical structures surrounding may be reduced. After careful planning, the precise robotic arm can stretch to hard-to-reach areas. The precise radiation delivered from the arm then minimizes the chance of injury to critical surrounding structures, with near-real-time image-guidance system eliminating the need for rigid immobilization, allowing robot arm 12 to track the body throughout the treatment.

Pre-Treatment Imaging and Treatment Planning

Typically, the target and its surrounding tissue are first imaged using CT, resulting in a volume of data. Other imaging modalities such as MRI, PET and ultrasound may also be used. The target volume is then delineated in this CT volume and a desired dose to the target is prescribed. Delicate or other tissue structures of concern in the vicinity of the target are also delineated and may be assigned a maximum desired dose that can be deposited at these structures. A computer program then receives the location and the shape of the target and the critical structures, the prescribed doses and the geometric configuration of the radiation delivery system and computes (a) the position and orientation of the beams to be fired and (b) a contour diagram showing dose received by all voxels in the CT volume. The radiation oncologist then reviews this data to see if the target is receiving the right dose and if structures in the vicinity receive too much dose. He or she may modify the boundaries of the target and the critical structures, along with dose received by them, to reach an acceptable treatment plan.

Treatment Delivery

During treatment delivery, the target can be first registered with the coordinate system of the treatment delivery system by using the patient registration system. The treatment delivery system may also receive the beam positions and orientations from the treatment planning stage. It then positions and orients the Linac and fires the beams towards the target.

Treatment Delivery in the Presence of Respiratory Motion

A preferred robot manipulator may be capable of positioning and orienting the Linac so that it follows the target due to breathing in real time. The ultrasound data acquired by the ultrasound imaging system in real-time or near real-time, registered with the CT volume provides real-time location of the target or the surrounding anatomical structures. If the ultrasound imaging is not real-time, a motion model may be used to assist targeting in real-time. The tracking system may first build a correlation model between the motion of the skin of the patient recorded by the LEDs mounted on the patient's chest and any fiducials implanted in the vicinity of the target and seen in the ultrasound data. (The tumor itself need not be visible in the X-rays). Alternatively, natural anatomical structures at or near the target may be used as fiducials. The correlation model can be built by taking a series of ultrasound data sets in quick succession for one or more breathing cycles and at the same time, recording the position of the skin using the signals from the LEDs. Ultrasound data may be intermittently acquired to verify the validity of the correlation model. If the model is no longer sufficiently valid, a fresh model is generated by following the same procedure as before.

Targets in the heart (tumors or other types of targets) poses two challenges for radiation delivery systems:

1. Implantation of fiducials in the heart muscle can be difficult and/or disadvantageous.

2. The heart itself beats fairly rapidly (for example, roughly at a rate of 1 beat every second), and some parts of the heart move more than the other parts due to this beating. In addition, the heart as a whole may also move or deform due to respiration.

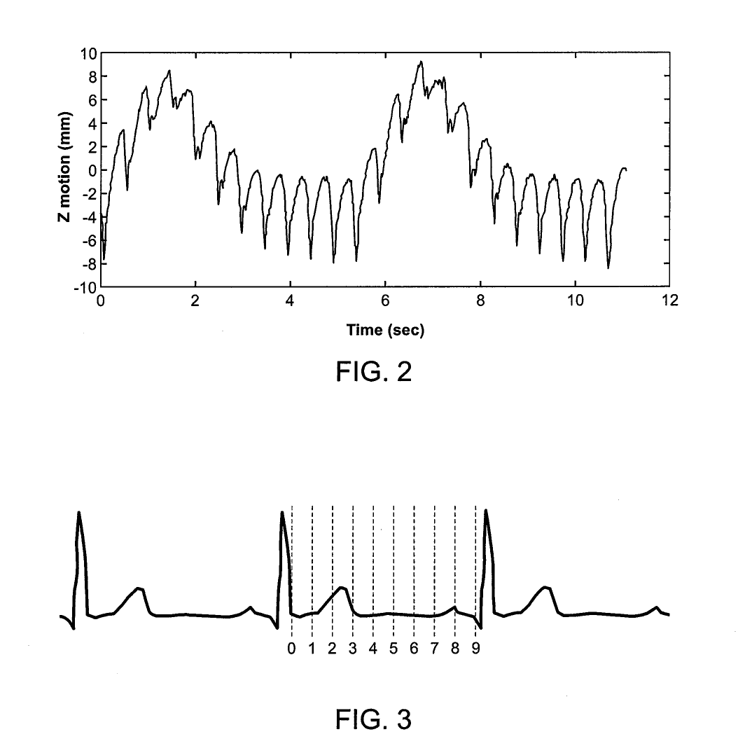

FIG. 2 shows the anterior/posterior motion of a point at the cavotricuspid isthmus inside the right atrium of a pig heart. As can be seen, the motion has two components: a slow varying breathing component and a rapidly varying cardiac component.

Embodiments of the present invention address either and/or both the above challenges and facilitates radiosurgery of targets in the heart muscle. Optionally, a beam of radiation may be redirected in response to a model or real-time ultrasound data including the target tissue or surrounding tissue, and/or a beam of radiation may be gated in response to the model or real-time ultrasound data.

Exemplary Method

1. Acquire a series of M CT volumes, CT(j), j=0, . . . , M-1, of the heart over one cardiac cycle with the patient holding his/her breath. Use a high speed CT scanner such as 64-slice Siemens SOMOTOM Definition to acquire CT volumes quickly, e.g. one volume in 83 ms. Contrast agents may be used.

2. FIG. 3 shows a typical EKG waveform with M=10 phases where 10 CT volumes are acquired. Outline the target in each of these M volumes. Alternatively, outline the target in one CT volume and automatically track it over all the CT volumes to generate the targets in other CT volumes.

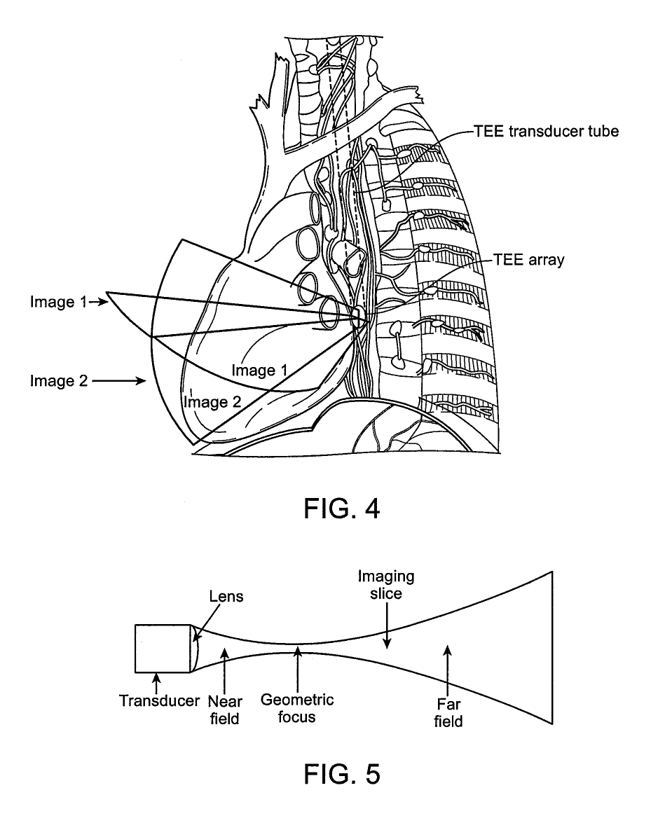

3. During treatment delivery, acquire an image of the target area and/or the surrounding 30 tissue using an ultrasound transducer. FIG. 4 shows an exemplary transducer, a transesophageal echo (TEE) ultrasound transducer in action. It is inserted into the esophagus and capable of acquiring ultrasound data, for example at least two orthogonal planes of ultrasound images simultaneously. Alternatively, transthoracic echo (TTC) ultrasound transducers or intracardiac echo (ICE) may also be used. Such a transducer may be capable of acquiring images at 30 frames per second or faster, a rate fast enough to visualize the moving heart. Slower frames per second may also be used. TEE bi-planar transducers are manufactured by Philips Medical Systems, Siemens Medical System and Others. Such transducers include two different types of transducers: (a) two or more ID array transducers affixed to a base such that the transducers have a fixed relationship to each other. For example two ID transducers may be 90 degrees to each other. In this case, the ID transducers are acquiring 2D slices through the heart. (b) A 2D array transducer capable of acquiring imaging planes that are 90 degrees to each other by using electronic steering. The ultrasound transducer is also fitted with a position sensor capable of measuring the position and orientation of the ultrasound transducer in 6 degrees of freedom (DOF). Aurora system, available from NDI, Waterloo, Ontario, Canada, is one such position sensor.

4. During treatment delivery, acquire ultrasound image pairs continuously. (An operator may initially manipulate transducer handle from the radiation-proof control room to orient the transducer properly so that good quality images of the heart are acquired. This can be done at the outset. Since the patient is lightly sedated and hence the patient movement during treatment is minimal, the transducer will stay in contact.) The system record both position and orientation of the transducer and also the EKG waveform. Acquire the following data in real time:
Ultrasound image pairs: US
Position and orientation of the ultrasound transducer, using the position sensor: P
EKG samples: EKG

5. For each ultrasound image pair, US, first determine cardiac phase by comparing the time stamp of the ultrasound image pair with the time stamps of the EKG data samples. This method is called retrospective gating. Alternatively the ultrasound images can be acquired at a series of pre-defined phases of the cardiac cycle by using prospective gating, where the ECG waveform is continuously analyzed by a system module and ultrasound images are acquired when the ECG waveform reaches anyone of the pre-defined cardiac phases.

6. Then register US, with the CT by using the CT volume from the corresponding cardiac phase. This registration step has the following stages:
Start with an initial position and orientation, Q, of the ultrasound image pair relative to the CT volume.
Simulate two orthogonal thick CT slices, called DRUS, corresponding to Q. FIG. 5 shows the slice thickness of a typical 2D ultrasound image. As can be seen, due to acoustic focusing methods used in a typical ultrasound transducer, the slice thickness is large near the face of the transducer, small at the geometric focus and large again in the far field. When simulating thick CT slices corresponding to Q, use the depth-dependent CT slices, by adding CT voxel values in the slice. The goal is to make the CT slices and ultrasound slices look similar. To accomplish this, other types of depth- and steering angle-dependent effects, such as the effects on ultrasound resolution, can also be simulated when generating the CT slice.
Optionally, pre-process US and/or CT volume or a part thereof, using techniques such as:
   Filtering (thresholding, gradient detection, curvature detection, edge enhancement, image enhancement, spatial frequency-based adaptive processing).
   Segmentation
   Mapping, such as windowing, nonlinear mapping
   Histogram equalization
   Spatial windowing, such as region-of-interest
   Higher order processing, such as connectivity model
   Multi-spectral processing
   Multi-scale processing
   Temporal processing, such as filtering, convolution, differentiation, integration, motion analysis and optical flow
Compute a similarity measure between the two DRUSs and the two orthogonal ultrasound images.
Repeat the process, near a neighborhood of the first value of Q, and pick the Q with an acceptable and/or the optimum similarity as the correct position and orientation of the ultrasound transducer relative to CT.

7. Transform the target location from CT to the coordinate system of the ultrasound transducer by using the registration step in #5 above.

8. Transform the target location from the coordinate system of the ultrasound transducer to the coordinate system of the treatment delivery system using data from the position sensor attached to the ultrasound transducer.

9. Fire the radiation beam to the target.

It will often be advantageous to acquire ultrasound data at a given cardiac phase from a sufficiently large number of locations that are not co-planar and also to know the position of those locations in the coordinate system of the robot. To achieve this, one or more other types of ultrasound transducers that have position sensors attached to them can also be used. These include: transthoracic, intracardiac, rotating, rocking, sliding, side-fired, forward looking, piezoelectric transducer (PZT), capacitive micromachined ultrasonic transducer (CMUT), 1D-arrays, 2D-arrays, and other types of ultrasonic transducers.

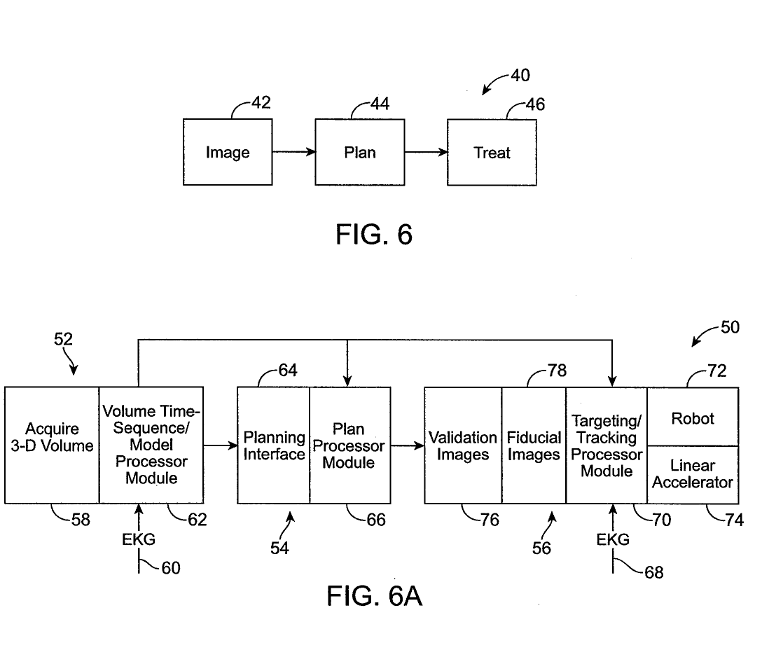

Referring now to FIGS. 6 and 6A, a relatively simple treatment flowchart 40 can represent steps used before and during radiosurgical treatment according to embodiments of the present invention. The internal tissues are imaged 42, typically using a remote imaging modality such as computed tomography (CT), magnetic resonance imaging (MRI), ultrasound imaging, X-ray imaging, optical coherence tomography, a combination of these or other imaging modalities, and/or the like. Note that the tissue structure which will be targeted need not necessarily be visible in the image, so long as sufficiently contrasting surrogate imagable structures are visible in the images to identify the target tissue location. The imaging used in many embodiments will include a time sequence of three dimensional tissue volumes, with the time sequence typically spanning one or more cycles (such as a cardiac or heartbeat cycle, a respiration or breathing cycle, and/or the like).

Based on the images, a plan 44 will be prepared for treatment of the target tissue, with the plan typically comprising a series of radiation beam trajectories which intersect within the target tissue. The radiation dose within the target tissue should be at least sufficient to provide the desired effect (often comprising ablation of tissue, inhibition of contractile pathways within the heart, inhibition of arrhythmogenesis, and/or the like). Radiation dosages outside the target tissues will decrease with a relatively steep gradient so as to inhibit damage to collateral tissues, with radiation dosages in specified sensitive and/or critical tissue structures often being below a desired maximum threshold to avoid deleterious side effects. Embodiments of the invention may employ the 3-D volumes acquired in the imaging step 42 during the planning 44, with exemplary embodiments making use of the motion model represented by the time sequence of 3-D tissue volumes so as to more accurately identify exposure of radiation outside of the target, within sensitive tissue structures, inside the target, and the like. Planned timing of some or all of a series of radiation beams may be established based on the cardiac cycle, the respiration cycle, and/or the like so as to generate the desired dosages within the target tissue, so as to minimize or inhibit radiation exposure to critical structures, and/or to provide desired gradients between the target tissue and collateral or sensitive structures. In some embodiments, the order of the planned radiation beams may be altered and/or the trajectories of the radiation beams may be calculated in response to the motion of the model volume.

Once the plan 44 is established, the treatment 46 can be implemented. The treatment will often make use of a processor to direct movement of a robotic structure supporting a radiation beam source, along with registration, validation, and/or tracking modules which enhance accuracy of the treatment. Tracking may employ the motion model developed during imaging 42, and/or may also employ a separate intra-operative motion model. Alternatively, no motion model will be used, instead the target location computed form the real-time ultrasound data will be used for tracking. The treatment 46 step and the associated hardware may use a sensor and/or input for physiological wave forms such as the respiration phase, cardiac phase, and the like for use in such tracking.

Referring to the exemplary simplified functional block diagram 50 of FIG. 6A, imaging 52, planning 54, and treatment 56 steps and/or structures are reflected (with slightly more detail) in the structure of the system provided to treat the heart. Imaging 52, planning 54, and treatment 56 structures are employed, with each structure including an associated processor module. The processor modules will typically comprise computer processing hardware and/or software, with the software typically being in the form of tangible media embodying computer-readable instructions or code for implementing one, some, or all of the method steps described herein. Suitable tangible media may comprise a random access memory (RAM), a read-only memory (ROM), a volatile memory, a non-volatile memory, a flash memory, a magnetic recording media (such as a hard disk, a floppy disk, or the like), an optical recording media (such as a compact disk (CD), a digital video disk (DVD), a read-only compact disk, a read/write compact disk, a memory stick, or the like). The various modules described herein may be implemented in a single processor board of a single general purpose computer, or may be run on several different processor boards of multiple proprietary computers, with the code, data, and signals being transmitted between the processor boards using a bus, a network (such as an Ethernet, intranet, or internet), via tangible recording media, using wireless telemetry, or the like. The code may be written as a monolithic software program, but will typically comprise a variety of separate subroutines and/or programs handling differing functions in any of a wide variety of software architectures, data processing arrangements, and the like. Nonetheless, breaking the functionality of the program into separate modules is useful for understanding the capabilities of the various aspects of the invention.

Addressing the imaging block 52 of block diagram 50 in FIG. 6A, a time-sequence of 3-D volumes may be acquired 58 as described above. Corresponding EKG signals 60 may also be received by the model processor module 62, and the processor may optionally use the EKG signals to time the acquisition of the 3-D volumes. In other embodiments, the respiratory signal may also be received by the model processor module 62, and the processor may optionally use the respiratory signal to time the acquisition of the 3D volumes. The series of radiation beams are planned, typically by a surgeon using a user interface 64 (such as a display and keyboard, mouse, or other input device) to communicate with a plan processor module 66. The processor module may make use of the model (including the tissue movements) to determine dosages in the target, collateral, and critical or sensitive tissues.

Once the patient is positioned for treatment relative to the treatment structure 56, an EKG sensor is coupled to the patient to provide EKG signals 68 to the targeting processor module 70. The targeting module configures the robot 72 so as to position and orient the linear accelerator 74 (or other radiation source) toward the target tissue along the desired trajectory for a particular radiation beam from among the series. Once the moving target tissue and the beam trajectory are appropriately aligned, the tracking module 70 may fire the radiation beam by energizing the linear accelerator 74. Hence, the tracking module benefits from the motion model developed during the imaging steps, and the model may optionally be revised using data obtained immediately before and/or during treatment.

Registration and validation of tracking may be provided using ultrasound data or the like from a remote image capture system 76, with the exemplary data being provided by a real-time biplanar TEE ultrasound imaging transducer and a position sensor attached to the transducer. Additionally, if the ultrasound data acquisition is not real-time, tracking of respiration-induced movement and the like may be provided using surface image capture devices 78 such as cameras, infrared cameras, or the like to generate signals indicating movement of surface fiducials. Input from the ultrasound imaging system 76 and surface image system 78 is also received by the tracking processor module 70.

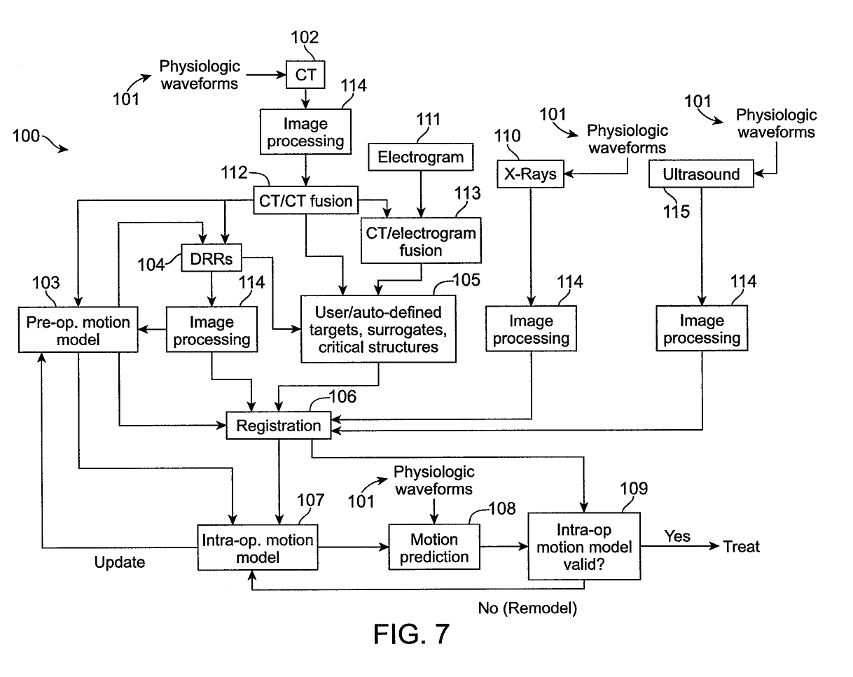

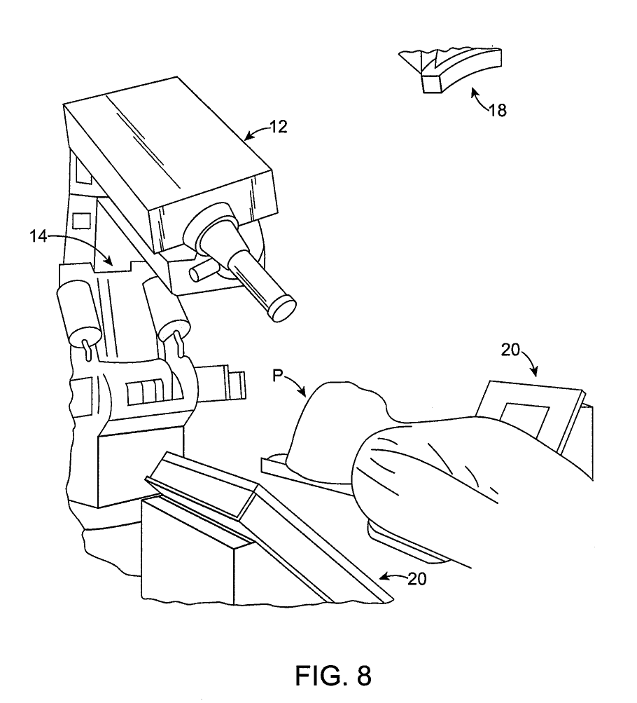

A more comprehensive functional block diagram of an exemplary heart treatment system 100 is schematically illustrated in FIG. 7. System 100 generally registers a series of radiation beams with a target despite motion of the target. The target will typically comprise an anatomical structure toward which the series of beams converge so as to deposit radiation therein. If the target is to view in ultrasound data or other remote imaging modalities, the system may employ surrogate structures, which may be anatomical structures visible in the ultrasound data near the target which can be aligned and tracked. The surrogate structure may alternatively be the same as the target. Alignment generally encompasses the act of registering the CT coordinate system (of a volume acquired from the patient) to the room coordinate system of the treatment system. Tracking encompasses the act of determining the target coordinates in the room coordinate system using, for example, ultrasound data. Other types of imaging, such as fluoroscopy or X-rays such as those used in the traditional CyberKnife system may also be used.

The target will generally have motion which includes two components: respiratory motion and cardiac motion. Similarly, the surrogate structure may have two motion components: respiratory motion and cardiac motion.

Referring to the individual components shown in FIG. 7, the physiological wave forms may include ECG signals and respiratory signals (including those derived from images of movement of LEDs or other surface fiducials). CT volumes 102 encompass a variety of different types of CT volumes, and may employ multiple types of CT volumes for a single patient. The CT volumes may be acquired at specific points along the cardiac cycle, respiration cycle, or the like.

Once all the desired CT volumes have been acquired, 2-D and/or 3-D image processing 114 of the acquired images or volumes may be employed. The image processing may include filtering, morphological filtering, mapping, gamma correction, connectivity mapping, distance mapping, order detection, ridge detection, curvature mapping, adaptive filtering, image enhancement, band pass filtering, unsharp mask filtering, top hat filtering, multi-spectral processing, multi-scale processing, and/or the like. Many of the acquired volumes may include a series of discrete images at different locations, so that a wide variety of 2-D image filtering and image processing techniques may be employed on the acquired volumes.

Some or all of the acquired CT volumes are fused 112, so that they are registered to a common reference frame. The common reference frame may be based on an anatomical structure such as the spine. Alternatively, deformable registration may be employed, or point-based registration may be used.

An electrogram 111 of a portion or all of the patient's heart may be obtained, and may be fused 113 with the acquired CT volumes. The electrogram may include a voltage map, an activation map, or the like, and may be acquired using commercially available systems such as the Carto™ system commercialized by Biosense Webster (a Johnson & Johnson company). Fusion of the CT volumes with the electrogram can effectively superimpose the electrogram data with the 3-D information in the CT volume and/or the 4-D information in the motion model, allowing (for example) treatment to be directed toward specific anatomical structures based in part on their mapped activation potentials. Alternatively, the electrogram may be superimposed with the ultrasound data 115 as well.

DRUSs 104 are generated from the CT volumes using any of a variety of techniques. The DRUSs will often correspond in orientation and location to ultrasound data 115 obtained by the treatment system while the patient is positioned for treatment. The ultrasound data 115 may be obtained at desired phases of physiological wave forms 101, and in addition may comprise fluoroscopic X-rays or other planar X-ray imaging types, with the X-rays typically being acquired from two or more views simultaneously, such as in the bi-planar X-ray system of the CyberKnife radiosurgical system.

In the planning stage, the system user and/or processor defines targets, surrogates, and critical or sensitive structures using the acquired CT volumes, the DRUSs, the electrograms, ultrasound data, and/or other available input. A pre-treatment motion model 103 may be generated using the acquired CT volumes, the images of the DRUSs, or other two or three dimensional information about the target and surrounding anatomy. The motion model 103 also employs the physiological wave forms 101, and most often the cardiac and/or respiratory phase information associated with each of the acquired 3-D volumes. A parametric motion model may be fitted to the data, or the raw data itself may be used so as to produce a lookup table (where the input is one or more physiological wave forms, and the output is the motion or a quantity derived from the motion such as position, velocity, acceleration, or the like for a given anatomical location in the 3-D space of the model volume or within a 2-D planar space corresponding to the DRUS). The pre-treatment motion model 103 may be applied to the CT volume data to generate a new DRUS. The DRUSs may, for example, have a desired associated cardiac phase, respiration phase, or the like.

Registration 106 encompasses registering the DRUSs and the ultrasound data, with or without use of the pre-treatment motion model. Registration may be a rigid registration or deformable registration, and may be using 1, 2, 3, 4, 5 or 6 dimensions. Registration could be separable, first performing the registration in a subset of dimensions, followed by registration in another subset of dimensions. Registration may also be a multi-scale registration. Registration 106 may employ multiple disjointed regions of interest (ROI) simultaneously. In exemplary embodiments, registration could be performed using different registration strategies, each fine-tuned to different ultrasound data sets. The results of the registration strategies could depend on the results of other registration strategies.

The preferred embodiment uses real-time ultrasound data and pre-acquired CT data for the purpose of targeting. If real-time ultrasound data is not available, an intra-operative motion model 107 may be used for targeting. The intra-operative motion model 107 will often employ the results of the pre-treatment motion model 103, together with the movement identified in the ultrasound data 115, X-ray images 110, and the like (often through matching of the surrogates) so as to describe the motion of the target and the sensitive structures with respect to the physiologic wave forms 101. The pre-treatment motion model 103 may be updated based on the information obtained as the system prepares for or implements the series of radiation beams using the intra-treatment motion model 107. Motion is predicted 108 using the intra-operative motion model 107 per the physiologic wave form signals 101, and the intra-operative motion model is validated 109 (typically by checking the predicted position and/or motion of the target or surrogate structures against the actual position and/or motion determined by the registration 106 of the most recent ultrasound data 115, X-ray images 110, and/or the like. If the model does not sufficiently accurately predict the motion and is thus not sufficiently valid, treatment may be interrupted, a new model may be built from scratch and/or the prior intra-operative model may be revised. If the model is within the desired threshold of accuracy, the treatment proceeds.

Referring now to FIGS. 6A and 7, CT volumes may be acquired (reference numerals 58 and 102) using a variety of different approaches. A cardiac gated CT volume may be acquired at a particular phase of the EKG cycle. Two variations of cardiac gated CT may include a held-breath version and a free-breathing version. In the held-breath cardiac gated CT, the patient is holding their breath (typically either at full inspiration or full expiration), so that respiration motion is absent while the data is acquired. In the free breathing cardiac gated CT, the patient is breathing freely. The CT volume may be acquired at a desired point of the respiration cycle. By measuring the respiration wave form, the exact respiratory phase at which the CT volume is acquired can be known (similar to the known cardiac phase at which the CT volume is acquired). In either variation, both the cardiac phase and the respiration cycle phase can be identified for the cardiac gated CT.

A cardiac gated 4-dimensional CT can be generated by acquiring a time series of cardiac gated CT volumes at a series of desired EKG phases. Once again, the 4-D cardiac gated CT can be a held-breath type or a free-breathing type (as described above). Additionally, regarding the free-breathing cardiac gated 4D CT, the resulting series of CT volumes may be acquired at the same EKG phase, typically throughout the respiration cycle. By associating each CT volume with the associated phase of the respiration cycle, the time series CT volumes can be used to model respiratory-induced motion of tissue while minimizing the cardiac motion artifacts.

Yet another type of volume which may be acquired is the respiratory-gated CT volume. Such CT volumes may be acquired at a particular phase of the respiration cycle. The cardiac motion may generally be ignored in this type of CT volume, so that the rapidly moving cardiac structures may be blurry in such CT volumes. In a related respiratory-gated 4-D CT volume, a series of respiratory-gated CT volumes are acquired at a series of respiratory phases.

DRUSs may be generated by simulating an ultrasound data set at a desired imaging plane or volume by modeling ultrasound data from the CT volume.

Still further improvements in the DRUSs may be provided, including the removal of bony anatomy, deformable registration of CT data with ultrasound data, and the like.

An exemplary patient treatment methodology may clarify the systems and methods described above. In an exemplary treatment, the patient may be treated for atrial flutter although many of the steps to be described may also be applicable to treatment of atrial fibrillation and other arrhythmias. In this embodiment, an anatomical target corresponding to a site of arrhythmogenesis may be chosen for ablation. Such ablation of an anatomic area in the heart can interrupt aberrant pathways or destroy a focus responsible for the arrhythmia. If an electrical map is available outlining abnormal conduction (such as an electrogram using the Carto™ system) the electrical map is correlated to an anatomic site within the heart.

A catheter is placed from a percutaneous venipuncture to the interior of the right atrium under fluoroscopic guidance. In the case of atrial flutter, the catheter may be positioned in or near the ostium of the coronary sinus. This structure is anatomically close (roughly about 1 cm) to the cavotricuspid isthmus, which is often the site of generation of atrial flutter rhythms. The ostium of the coronary sinus may also move in correlation with the cavotricuspid isthmus. Alternatively, a catheter can be placed deep in to the coronary sinus. One or more fiducials can also be placed on such catheter. Another catheter may be separately placed via venipuncture and positioned directly on the target cavotricuspid isthmus if desired. Each catheter will have an imagable material near the associated anatomy to be targeted or used as a surrogate structure functioning as a fiducial. The electrodes in ablation catheters may also function as fiducials. This coronary sinus structure can be accessed with an appropriate catheter tip and moves synchronously with the target in three dimensions, so that knowing the position of such a surrogate fiducial catheter allows one to accurately target the desired anatomical structure. The catheter is visible within the CT volume and the ultrasound data, and can be removed after treatment. Catheters and hence the fiducials can also be temporarily affixed to the cardiac tissue by mechanical means such as using a screw. A cardiac pacing lead is an example.

A CT scan is performed using both cardiac and respiratory-gating so as to obtain a 3-D motion model corresponding to cardiac cycle movement, respiration cycle movement, and/or both. The CT data is fed into the treatment planning module, allowing a library of images to be viewed and the target volume to be identified in three dimensions.

An electrophysiologist and/or cardiologist (for example, the treatment planning physician) may work with a radiation oncologist to generate a treatment plan that deposits radiation with the desired dose at the targeted area (in our example in the cavotricuspid isthmus) so as to inhibit atrial flutter. The radiation dose will result in ablation of the myocardium and will interfere with the abnormal pathway or focus of the arrhythmia. The prescribed dose will typically be in a range from about 15 to about 80 Gy to achieve the desired ablation, and the ablated region may be planned conformably (with consideration of a concentric deposition of dose around an isocenter) or non-conformably (to adjust the dose shape deposited to avoid nearby critical or sensitive structures that the treating physician(s) desires to avoid exposing to excessive radiation). The treatment plan may be reviewed for (among other considerations) the dose, the targeted anatomy, avoidance of critical or sensitive structures near the target, or through which radiation beams should not pass, modification of treatment to the target based on consideration of the motion at the target (based on respiratory and/or cardiac cycle contributions) and/or the like.

The treatment plan is transmitted into the treatment system, and the patient is positioned on the treatment table. Respiratory cycle indicators such as sensors or LEDs can be placed on the chest wall of the patient to provide information (optionally via surface imaging) to the treatment system regarding chest wall motion. The treatment system processor module may predict and/or verify the motion of the target and/or surrogate structures by identifying the respiratory cycle using an intra-treatment model as described above. The patient may also have cutaneous electrocardiogram electrodes placed such that the treating physician and treatment processor module can monitor the cardiac rhythm that the patient is undergoing during treatment.

The treatment takes place by configuring the robot and energizing the radiation source per the series of radiation beams that have been planned. The patient may be monitored via closed circuit TV and/or using sensors such as heart rate monitors, blood pressure monitors, and other biosensors for any changes during treatment. At the completion of treatment, cutaneous sensors and catheters can be removed. The patient's cardiac rhythm may be monitored remotely via telemetry during a follow-up period.

As illustrated in FIG. 8, during a procedure with system 10, a patient P lies still on a treatment table. Light sedation may be used when using a TEE ultrasound transducer. Generally no sedation or anesthesia is used because the treatment is painless, and the procedure can last anywhere from between 30 to 90 minutes depending on the complexity of the case and the dose to be delivered. The treatment itself involves the administration of numerous radiation beams delivered from different directions, typically in 10 to 15 second bursts.

Advantageously, the treatments described herein can be iterative. Rather than target many foci or regions as is often done in an invasive procedure, externally applied radiosurgical ablation can address one or more target shapes on one day, and the then other target shapes on another day as needed. The interim period between treatments can be used to access the need for subsequent treatments. Such iterative or fractionated treatment is thus more conservative than current methods.

Suitable types of radiation, including particle beam radiation, may be employed. For example, the present invention encompasses the use of a GammaKnife™ radiosurgery system to ablate the moving tissue. Although gamma radiation could be administered during open heart or other invasive procedures, the currently preferred applications are substantially non-surgical.

All suitable radiosurgery systems are contemplated with the energy source, duration and other parameters varying according to a size of the patient and other factors. A typical GammaKnife™ radiosurgery system may contain (for example) at least about 200 cobalt-60 sources of approximately 30 curies each, all of which are placed in an array under a heavily shielded unit. The shielded unit preferably comprises lead or other heavy metals.

While the exemplary embodiments have been described in some detail, by way of example and for clarity of understanding, those of skill in the art will recognize that a variety of modification, adaptations, and changes may be employed. Hence, the scope of the present invention should be limited solely by the appending claims.

What is claimed is:

1. A method for treating a moving tissue, the method comprising:
acquiring at least one volume of the tissue;
acquiring at least one ultrasound data set, image or volume of the tissue using an ultrasound transducer disposed at a position;
computing a similarity measure between:
the ultrasound data set, image or volume and
the acquired volume or a simulated ultrasound data set, image or volume therefrom;
configuring a robot in response to the similarity measure and the position of the transducer; and
firing a radiation beam from the configured robot.

2. The method of claim 1, wherein firing a radiation beam includes firing a radiation beam towards a target on a moving heart

3. The method of claim 1, wherein acquiring at least one volume of the tissue includes acquiring at least one CT volume of the tissue.

4. The method of claim 3, wherein the at least one CT volume of the tissue comprises a series of CT volumes of the tissue over a cycle, and wherein acquiring at least one ultrasound data set, image or volume of the tissue includes a series of acquiring ultrasound image pairs or volume and associated position information of the tissue over the cycle.

5. The method of claim 4, further comprising:
determining the part of the cycle for each ultrasound image pair or volume; and
registering each ultrasound image pair or volume with the series of CT volumes of the tissue by using the CT volume from the corresponding phase of the cycle.

6. The method of claim 4, wherein the cycle is a cardiac cycle, a respiratory cycle, or a combination of both cardiac and respiratory cycles.

7. The method of claim 1, wherein the transducer is capable of acquiring images or volumes at 30 frames per second or faster.

8. The method of claim 1, wherein a position sensor coupled to the transducer measures at least the position and orientation of the transducer in 6 degrees of freedom (DOF).

9. The method of claim 1, wherein computing a similarity measure between the ultrasound data set, image or volume and the acquired volume includes transforming at least one volume into the coordinate system of the transducer.

10. A method as in claim 1, wherein the transducer is selected from the group consisting of transesophageal (TEE), transthoracic, intracardiac, rotating, rocking, sliding, side-fired, forward looking, piezoelectric transducer (PZT), capacitive micromachined ultrasonic transducer (CMUT), 1D-arrays, and 2D-arrays.

11. A method for treating a heart, the method comprising:
acquiring at least one volume of the heart;
acquiring at least one ultrasound data set of the heart using a transducer;
simulating an ultrasound data set from the volume;
computing a similarity measure between the ultrasound data set and the simulated ultrasound data set;
configuring a robot dependent on the similarity measure; and
firing a radiation beam dependent on the configuration of the robot.

12. The method of claim 11, wherein simulating ultrasound data from the volume includes simulating depth-dependent effects of ultrasound image formation.

13. The method of claim 11, wherein simulating ultrasound data from the volume includes simulating depth-dependent resolution.

14. The method of claim 11, wherein simulating ultrasound data from the volume includes simulating steering angle-dependent effects of ultrasound image formation.

15. The method of claim 11, wherein simulating ultrasound data from the volume includes simulating steering angle-dependent resolution.

16. The method of claim 11, wherein the at least one volume of the heart comprises a series of CT volumes of the heart over a cycle, and wherein acquiring at least one ultrasound image or volume of the heart includes acquiring a series of ultrasound image pairs and associated position information of the heart over the cycle.

17. The method of claim 16, further comprising:
determining the part of the cycle for each ultrasound image pair; and
registering each ultrasound image pair with the series of CT volumes by using the CT volume from the corresponding phase of the cycle.

18. The method of claim 16, wherein the cycle is a cardiac cycle, a respiratory cycle, or a combination of both cardiac and respiratory cycles.

19. The method of claim 11, wherein computing a similarity measure between the ultrasound data set, and the simulated ultrasound data set includes transforming at least one volume into the coordinate system of the transducer.

20. The method of claim 11, wherein acquiring at least one ultrasound data set of the heart includes acquiring at least two orthogonal planes of ultrasound images of the heart.

21. The method of claim 20, further comprising determining a cardiac phase for each ultrasonic image pair.

22. The method of claim 11, further comprising acquiring an EKG waveform.

23. The method of claim 22, wherein acquiring at least one volume of the heart includes acquiring a CT volume of the heart at a particular cardiac phase of the EKG waveform.

24. The method of claim 22, wherein acquiring at least one ultrasound data set of the heart includes acquiring an ultrasound data set of the heart when the EKG waveform reaches a predefined phase.

25. The method of claim 11, wherein the transducer is capable of acquiring images at a rate fast enough to visualize a moving heart.

26. The method of claim 11, wherein the transducer is capable of acquiring images at 30 frames per second or faster.

27. The method of claim 11, wherein a position sensor measures at least the position and orientation of the transducer in 6 DOF.

28. A method as in claim 11, wherein the transducer is selected from the group consisting of transesophageal (TEE), transthoracic, intracardiac, rotating, rocking, sliding, side-fired, forward looking, piezoelectric transducer (PZT), capacitive micromachined ultrasonic transducer (CMUT), 1D-arrays, and 2D-arrays.

29. A method for treating a target on a moving heart, the method comprising:
acquiring at least one volume of the target;
acquiring at least one ultrasound volume of the target using a volumetric transducer;
computing a similarity measure between the ultrasound volume and the volume;
configuring a 6 DOF robot dependent on the similarity measure and on a position of the transducer; and
firing a radiation beam at the target dependent on the configuration of the robot.

30. The method of claim 29, wherein the target is located in the heart.

31. The method of claim 29, wherein the at least one volume of the target is a series of CT volumes of the target over a cycle, and wherein acquiring at least one ultrasound volume includes acquiring a series of ultrasound volume of the target and associated position information over the cycle.

32. The method of claim 31, further comprising:
determining the part of the cycle for each ultrasound volume; and
registering each ultrasound volume with the series of CT volumes by using the CT volume from the corresponding phase of the cycle.

33. The method of claim 31, wherein the cycle is a cardiac cycle, a respiratory cycle, or a combination of both cardiac and respiratory cycles.

34. The method of claim 29, wherein computing a similarity measure between the ultrasound volume and the CT volume includes transforming the at least one CT volume into the coordinate system of the transducer.

35. A method as in claim 29, wherein the transducer is selected from the group consisting of transesophageal (TEE), transthoracic, intracardiac, rotating, rocking, sliding, side-fired, forward looking, piezoelectric transducer (PZT), capacitive micromachined ultrasonic transducer (CMUT), 1D-arrays, and 2D-arrays.

36. The method of claim 29, further comprising acquiring an EKG waveform.

37. The method of claim 36, wherein acquiring at least one CT volume includes acquiring a CT volume at a particular cardiac phase of the EKG waveform.

38. The method of claim 36, wherein acquiring at least one ultrasound volume includes acquiring an ultrasound volume when the EKG waveform reaches a predefined phase.

39. The method of claim 29, wherein the transducer is capable of acquiring ultrasound volumes at a rate fast enough to visualize the moving heart.

40. The method of claim 29, wherein the transducer is capable of acquiring ultrasound volumes at 30 frames per second or faster.

41. A system for treating a moving tissue, the system comprising:
a volume acquisition system for acquiring at least one volume of the tissue;
a transducer with a position sensor for acquiring at least one ultrasound data set, image or volume of the tissue and associated position information;
a processor coupled to the acquisition system and the transducer, the processor configured for computing a similarity measure between:
the ultrasound data set, image or volume and
the acquired volume or a simulated ultrasound data set, image or volume therefrom;
a robot coupled to the processor so as to be configured in response to the similarity measure and data and the associated position information; and
a radiation source supported by the robot.

42. The system of claim 41, wherein the volume acquisition system is capable of acquiring a plurality of volumes of the tissue over a cycle.

43. The system of claim 41, wherein the transducer is capable of acquiring a plurality of images or volumes of the tissue and associated position information over a cycle.
